(19) United States
(12) Patent Application Publication
(10) Pub. No.: US 2013/0296447 A1
(43) Pub. Date: Nov. 7, 2013

(54) Method of Preventing Neurodevelopmental Damage Due to Prenatal Exposure to Environmental Toxins

(71) Applicant: James Alexander Bralley, III, Johns Creek, GA (US)

(72) Inventor: James Alexander Bralley, III, Johns Creek, GA (US)

(21) Appl. No.: 13/830,602

(22) Filed: Mar. 14, 2013

Related U.S. Application Data
(60) Provisional application No. 61/610,921, filed on Mar. 14, 2012.

(51) Int. Cl. C12Q 1/68

(52) U.S. Cl. CPC C12Q 1/6883
USPC 514/789; 435/6.11

(57) ABSTRACT

A method of assessing the risk in female patients of having neurocognitive developmentally disabled children. The method including measuring the toxin concentrations in the urine of a female subject wishing to become pregnant, or of a subject that is pregnant. Quantify the risk of neurocognitive developmentally disabled children born to the subject by comparing the toxin concentrations in the subject to reference concentrations utilizing a weighting algorithm. Advise the patient of the quantified risk of having neurocognitive developmentally disabled children. The concentrations of dialkyl phosphates, endocrine disruptors, and xenoestrogens compounds are evaluated. Additionally, the level of PON1 activity may be included in the risk assessment weighting algorithm.

### CROSS REFERENCE TO A PROVISIONAL APPLICATION

This application claims the benefit of Provisional Application Ser. No. 61/610,921 filed on Mar. 14, 2012, the entirety of which is hereby incorporated herein by reference.

### BACKGROUND OF THE INVENTION

1. Field of the Invention

The present invention relates to assessing the risk of female patients for having neurocognitive developmentally disabled children due to environmental toxins. Specifically, the concentrations of dialkyl phosphates, endocrine disruptors, and xenoestrogens compounds and PON1 activity are evaluated.

2. Background of the Invention

The following literature is of use in the subject matter of the present invention and is incorporated herein by reference:

1. Brouchard, M. F., et al., Prenatal Exposure to Organophosphate Pesticides and IQ in 7-Year-Old Children, Environmental Health Perspectives, 2011, 119(8): p. 1189-95.

2. Engel, S. M., et al., Prenatal Exposure to Organophosphates, Paraoxonase 1, and Cognitive Development in Childhood, Environmental Health Perspectives, 2011, 119(8): p. 1182-88.

3. Rauh, V. A. et al., Seven-Year Neurodevelopmental Scores and Prenatal Exposure to Chlorpyrifos, a Common Agricultural Pesticide, Environmental Health Perspectives, 2011, 119(8): p. 1196-1201.

There is an obvious need in the art for a process and method of assessing the risk of female patients for having neurocognitive developmentally disabled children due to environmental toxins. The toxins should include the exposure to organophosphate pesticides, endocrine disruptors, and xenoestrogens. A non-invasive means of measuring the environmental exposure of the female is via a urine sample. The invention may provide quantitative risk assessment score via an algorithm based upon the relative concentrations found for any combination of the environmental toxins. An evaluation of deficiency in PON1 activity may also be included in the risk assessment algorithm. The risk assessment algorithm may use specific weighting factors for each of the dialkyl phosphates, endocrine disruptors, and xenoestrogens compounds, and PON1 activity. It is to such a process and method of preventing neurodevelopmental damage due to prenatal exposure to environmental toxins that the present invention is primarily directed.

### SUMMARY OF THE INVENTION

In one aspect the present invention provides a method of assessing the risk of a female subject having neurocognitive developmentally disabled children. The method includes the steps of measuring toxin concentrations in the urine of a female subject wishing to become pregnant, or of a subject that is pregnant. Quantify the risk of neurocognitive developmentally disabled children born to the subject by comparing the toxin concentrations in the subject to reference concentrations utilizing a weighting algorithm.

In another aspect of the present invention, the toxin concentration measured includes the concentration levels in the subjects urine of the dialkyl phosphates: Dimethylphosphate (DMP), Dimethylthiophosphate (DMTP), Dimethyldithiophosphate (DMDTP), Diethylphosphate (DEP), Diethylthiophosphate (DETP), and Diethyldithiophosphate (DEDTP)3

In another aspect of the present invention, the toxin concentration measured includes the concentration levels in the subjects urine of the endocrine disruptors: Bisphenol A, Triclosan, and 4-Nonylphenol.

In another aspect of the present invention, the toxin concentration measured includes the concentration levels in the subjects urine of the xenoestrogens: mono-ethyl phthalate (MEtP),mono-2-ethylhexyl phthalate (MEHP), mono-(2-ethyl-5-hydroxyhexyl) phthalate (MEHHP), mono-(2-ethyl-5-oxohexyl) phthalate (MEOHP), methylparaben, ethylparaben, propylparaben, and butylparaben.

In another alternative aspect of the present invention, the method of assessing the risk of a female subject having neurocognitive developmentally disabled children further includes determining if the subject has a deficiency of PON1 activity by assessing the presence of a defective PON1 gene by single nucleotide polymorphism (SNP) detection. Utilizing the PON1 activity level in the weighting algorithm during the quantification of risk of neurocognitive developmentally disabled children.

In another alternative aspect of the present invention, the method includes determining effective amounts of nutritional supplementation that may reduce the subject's toxin concentrations towards the reference concentrations. Formulate a customized nutritional supplement containing the effective amounts of nutritional supplementation. Then, administer the nutritional supplementation to the subject over a period of time.

In other alternative aspect of the present invention, the method includes determining if the subject has a deficiency of PON1 activity by assessing the presence of a defective PON1 gene. The determination of the effective amount of nutritional supplementation is then affected by the level of PON1 activity. Quantify the risk of neurocognitive developmentally disabled children born to the subject subsequent to the nutritional supplementation.

These and other aspects of the invention will become apparent from the following description of the preferred embodiments taken in conjunction with the tables and figures. As would be obvious to one skilled in the art, many variations and modifications of the invention may be effected without departing from the spirit and scope of the novel concepts of the disclosure.

### DETAILED DESCRIPTION OF PREFERRED EMBODIMENTS

The present invention presents a method of assessing the risk of female patients for having neurocognitive developmentally disabled children due to environmental toxins. The concentrations of dialkyl phosphates, endocrine disruptors, and xenoestrogens are measured in the urine of pregnant women or those wishing to become pregnant. The invention also presents a quantitative risk assessment score via an algorithm based upon the relative concentrations found for any combination of the dialkyl phosphates, endocrine disruptors, or the analytes of xenoestrogens above. An evaluation of deficiency in PON1 activity is presented and may also be included in the risk assessment algorithm. The risk assessment algorithm may use specific weighting factors for each of the dialkyl phosphates, endocrine disruptors, and xenoestrogens compounds, and PON1 activity.

Studies have shown that pregnant women with the highest levels of organophosphate compounds in their bodies have children which have significant impairments of neurocognitive development. Residential exposure to organophosphate pesticides (“OP”) is still relatively common. In a sample of pregnant women in New York City in 2002, detectable levels of one OP was found in 100% of their indoor air samples and 70% of umbilical cord blood samples. Children of women with low paraoxonase activity (“PON1”) are particularly susceptible to neurodevelopmental problems. PON1 is an enzyme which metabolizes and detoxifies OPs in the body. Deficiencies in the activity of PON1 have been associated with increased toxicity of OP in these individuals.

Organophosphate pesticides are metabolized into the six dialkyl phosphates that show up in urine samples:
Dimethylphosphate (DMP)
Dimethylthiophosphate (DMTP)
Dimethyldithiophosphate (DMDTP)
Diethylphosphate (DEP)
Diethylthiophosphate (DETP)
Diethyldithiophosphate (DEDTP)3

Another group of everyday environmental toxins to which pregnant women may be exposed and which may result in children with significant impairments of neurocognitive development are know as endocrine disruptors. Endocrine disruptors weakly bind to estrogen receptors which can affect the endocrine, nervous, and immune systems as well as block thyroid hormone action within the mother. The three common endocrine disruptors which may be analyzed in urine samples:
Bisphenol A
Triclosan
4-Nonylphenol

Phthalates and parabens are often classified as xenoestrogens, foreign compounds in the body functioning as endocrine disruptors by binding specifically to estrogen receptors. Exposure to chemicals that are xenoestrogens disrupt the proper function of the body's endocrine system. Children and babies in the womb are most susceptible to hormonal and neurological development issues from exposure. The two common classes of xenoestrogens are Phthalates, also called “plasticizers,” and Parabens. The common analytes of xenoestrogens which may be analyzed in urine samples are:
mono-ethyl phthalate (MEtP)
mono-2-ethylhexyl phthalate (MEHP)
mono-(2-ethyl-5-hydroxyhexyl) phthalate (MEHHP)
mono-(2-ethyl-5-oxohexyl) phthalate (MEOHP)
methylparaben
ethylparaben
propylparaben
butylparaben

A step in the method of the present invention is to measure the OP concentrations in the urine of pregnant women or those wishing to become pregnant, collectively referred to herein as the patient, woman, or subject. Urine samples are collected from a subject and the level of dialkyl phosphates are measured with standard lab techniques. In an alternative embodiment of the present invention, another step is to determine if the patient has a deficiency in PON1 activity. A deficiency in PON1 activity is identified by looking for the presence of the defective PON1 gene in the patient by single nucleotide polymorphism (“SNP”) detection. The method then uses the OP concentration and may use the PON1 activity data to determine the relative risk the female patient has for having a child with adversely affected neurological development. The testing lab and/or consulting physician then inform the patient of this risk level and provide a plan for avoidance of OP. In an alternative embodiment of the present invention, a means is provided for supporting detoxification of OP in the woman through nutritional supplementation, low temperature saunas, and/or other means of detoxification methods as are known in the art. The method of the present invention can be done with or without PON1 SNP detection.

In another alternative embodiment of the present invention, a step of the method is to measure other toxins including endocrine disruptors, and/or the analytes of xenoestrogens concentrations in the urine of pregnant women or those wishing to become pregnant. As with OP toxins, in another alternative embodiment of the present invention, another optional step is to determine if the woman has a deficiency in PON1 activity. The method then uses the toxin concentration and may use the PON1 activity data to determine the relative risk the woman has for having a child with adversely affected neurological development. The testing lab and/or consulting physician then inform the patient of this risk level and provide a plan for avoidance of the problem toxins. In an alternative embodiment, a means is provided for supporting detoxification of the woman by nutritional supplementation, low temperature saunas, and/or other detoxification methods as are known in the art. The method of the present invention can be done with or without PON1 SNP detection.

In another embodiment, the present invention presents a multi-toxin quantitative test which includes any combination of; OP's, endocrine disruptors, and/or the analytes of xenoestrogens. The patient is assigned a quantitative risk assessment score via an algorithm based upon the relative concentrations found for any combination of the dialkyl phosphates, endocrine disruptors, or the analytes of xenoestrogens above. An initial weighting factor of each toxin is assigned. The weighting factor of each toxin will be updated as new research data is reflected back into the risk assessment algorithm. A defective PON1 gene is more critical for some toxins above than others. In another alternative embodiment of the invention, a unique weighting factor for each toxin may be used depending upon the PON1 activity data, and the results reflected in the quantitative patient score.

In another embodiment of the present invention, beneficial reductions in toxin concentrations can be determined by retesting patients after their consumption over time of the nutritional supplementation. In yet another embodiment, algorithms are developed to determine which nutritional supplementation best reduces toxin concentrations in the mother. Algorithms are developed for both normal and defective PON1 gene patients.

All of the compounds, processes and methods disclosed and claimed herein can be made and executed without undue experimentation in light of the present disclosure. While the compounds, processes and methods of this invention have been described in terms of preferred embodiments, it will be apparent to those of skill in the art that variations may be applied to the compounds, processes and methods and in the steps or in the sequence of steps of the methods described herein without departing from the concept, spirit, and scope of the invention.

More specifically, it will be apparent that certain compounds which are both chemically and physiologically related may be substituted for the compounds described herein while the same or similar results would be achieved. All such similar substitutes and modifications apparent to those skilled in the art are deemed to be within the spirit, scope, and concept of the invention.

What is claimed is:

1. A method of assessing the risk of a female subject having neurocognitive developmentally disabled children, the method comprising the steps of;
measuring organophosphate concentrations in the urine of a female subject wishing to become pregnant, or of a subject that is pregnant;
determine if the subject has a deficiency of PON1 activity by assessing the presence of a defective PON1 gene by single nucleotide polymorphism (SNP) detection; and
quantify the risk of neurocognitive developmentally disabled children born to the subject by at least one of; comparing the organophosphate concentration in the subject to a reference concentration, by the level of PON1 activity.

2. The method of claim 1, the method further comprising the steps of;
determining effective amounts of nutritional supplementation that may reduce the subject's organophosphate concentration towards the reference concentration;
formulating a customized nutritional supplement containing the effective amount of nutritional supplementation; and
administering the nutritional supplementation to the subject over a period of time.

3. The method of claim 2, wherein the effective amount of nutritional supplementation is affected by the level of PON1 activity.

4. The method of claim 2, further comprising the step of:
quantifying the risk of neurocognitive developmentally disabled children born to the subject subsequent to the nutritional supplementation.

5. A method of assessing the risk of a female subject having neurocognitive developmentally disabled children, the method comprising the steps of;
measuring toxin concentrations in the urine of a female subject wishing to become pregnant, or of a subject that is pregnant;
determine if the subject has a deficiency of PON1 activity by assessing the presence of a defective PON1 gene by single nucleotide polymorphism (SNP) detection; and
quantify the risk of neurocognitive developmentally disabled children born to the subject by at least one of; comparing the toxin concentrations in the subject to reference concentrations, by the level of PON1 activity.

6. The method of claim 5, wherein the toxin concentration measured includes at least one of: Dimethylphosphate (DMP), Dimethylthiophosphate (DMTP), Dimethyldithiophosphate (DMDTP), Diethylphosphate (DEP), Diethylthiophosphate (DETP), and Diethyldithiophosphate (DEDTP)3

7. The method of claim 5, wherein the toxin concentration measured includes at least one of: Bisphenol A, Triclosan, and 4-Nonylphenol.

8. The method of claim 5, wherein the toxin concentration measured includes at least one of: mono-ethyl phthalate (MEtP),mono-2-ethylhexyl phthalate (MEHP), mono-(2-ethyl-5-hydroxyhexyl) phthalate (MEHHP), mono-(2-ethyl-5-oxohexyl) phthalate (MEOHP), methylparaben, ethylparaben, propylparaben, and butylparaben.

9. The method of claim 5, the method further comprising the steps of;
determining effective amounts of nutritional supplementation that may reduce the subject's toxin concentrations towards the reference concentrations;
formulating a customized nutritional supplement containing the effective amounts of nutritional supplementation; and
administering the nutritional supplementation to the subject over a period of time.

10. The method of claim 9, wherein the effective amount of nutritional supplementation is affected by the level of PON1 activity.

11. The method of claim 9, further comprising the step of:
quantify the risk of neurocognitive developmentally disabled children born to the subject subsequent to the nutritional supplementation.

12. A method of assessing the risk of a female subject having neurocognitive developmentally disabled children, the method comprising the steps of;
measuring toxin concentrations in the urine of a female subject wishing to become pregnant, or of a subject that is pregnant; and
quantify the risk of neurocognitive developmentally disabled children born to the subject by comparing the toxin concentrations in the subject to reference concentrations utilizing a weighting algorithm.

13. The method of claim 12, wherein the toxin concentration measured includes at least one of: Dimethylphosphate (DMP), Dimethylthiophosphate (DMTP), Dimethyldithiophosphate (DMDTP), Diethylphosphate (DEP), Diethylthiophosphate (DETP), and Diethyldithiophosphate (DEDTP)3

14. The method of claim 12, wherein the toxin concentration measured includes at least one of: Bisphenol A, Triclosan, and 4-Nonylphenol.

15. The method of claim 12, wherein the toxin concentration measured includes at least one of: mono-ethyl phthalate (MEtP),mono-2-ethylhexyl phthalate (MEHP), mono-(2-ethyl-5-hydroxyhexyl) phthalate (MEHHP), mono-(2-ethyl-5-oxohexyl) phthalate (MEOHP), methylparaben, ethylparaben, propylparaben, and butylparaben.

16. The method of claim 12, the method further comprising the steps of;
determining if the subject has a deficiency of PON1 activity by assessing the presence of a defective PON1 gene;
utilizing the PON1 activity level in the weighting algorithm during the quantification of risk of neurocognitive developmentally disabled children.

17. The method of claim 12, the method further comprising the steps of;
determining effective amounts of nutritional supplementation that may reduce the subject's toxin concentrations towards the reference concentrations;
formulating a customized nutritional supplement containing the effective amounts of nutritional supplementation; and
administering the nutritional supplementation to the subject over a period of time.

18. The method of claim 17, the method further comprising the steps of;
determining if the subject has a deficiency of PON1 activity by assessing the presence of a defective PON1 gene;
wherein the determination of the effective amount of nutritional supplementation is affected by the level of PON1 activity.

19. The method of claim 17, further comprising the step of:
quantify the risk of neurocognitive developmentally disabled children born to the subject subsequent to the nutritional supplementation.
